(19) United States
(12) Patent Application Publication
(10) Pub. No.: US 2011/0097324 A1
(43) Pub. Date: Apr. 28, 2011

(54) COMPOSITIONS AND METHODS FOR MODULATING NICOTINIC/NMDA RECEPTOR FUNCTION

(75) Inventor: Fang Liu, Mississauga (CA)

(73) Assignee: CENTRE FOR ADDICTION AND MENTAL HEALTH, Toronto, ON (CA)

(21) Appl. No.: 12/997,820

(22) PCT Filed: Jun. 15, 2009
(86) PCT No.: PCT/CA09/00831

§ 371 Date: Dec. 13, 2010

(30) Foreign Application Priority Data
Jun. 13, 2008 (US) 61061445

(51) Int. Cl. A61K 39/395; A61P 25/30; A61K 31/7088; A61K 38/14; C07K 7/06; C07K 7/08; C07K 14/00; C07H 21/04; C12N 5/02

(52) U.S. Cl. 424/134.1; 424/139.1; 514/44 R; 514/20.9; 530/329; 530/328; 530/327; 530/326; 530/325; 530/324; 530/350; 536/23.1; 435/375

(57) ABSTRACT

The present invention provides a method for modulating nicotinic/NMDA receptor function in a mammal in need of such treatment comprising administering a therapeutically effective amount of an agent that disrupts heterodimerization of α7 neuronal nicotinic acetylcholine receptors and N-methyl-D-asparate glutamate receptor. A polypeptide and fragments thereof comprising an amino acid sequence selected from the second intracellular loop of the α7 nAchR and carboxyl tail of the N-methyl-D-aspartate receptor are also provided, which are able to inhibit the heterodimerization. Also disclosed are nucleotide sequences encoding the polypeptides, and methods of inhibiting the heterodimerization of α7 nAchR and NMDAR using the polypeptides and nucleic acids.

### FIELD OF THE INVENTION

The present invention relates to compositions and methods for modulating nicotinic/NMDA receptor function. In particular, the present invention relates to compositions and methods for modulating the heterodimerization of α7 nicotinic acetylcholine receptors (nAChRs) and N-methyl-D-aspartate (NMDA) glutamate receptors.

### BACKGROUND OF THE INVENTION

The market for smoking cessation therapeutics is growing due to a rise in public awareness of dangers of smoking, legislation banning smoking, a need for more effective treatments, and development of novel therapies.

Currently, there is a lack of product differentiation on the market, with the types of treatments having limited product efficacy. Nicotine replacement therapies focus on easing withdrawal symptoms but present considerable health risks if users continue to smoke. Long term (one year) smoking cessation rates are low and vary from 5% to 20% with this form of treatment. There are two medical alternatives to nicotine replacement therapies licensed as smoking cessation aids, buproprion, marketed as Zyban® by GlaxoSmithKline, and varenicline, marketed as Chantix® by Pfizer. Buproprion is primarily an antidepressant but it's been effective in reducing nicotine withdrawal symptoms. However, the precise mode of action on dopaminergic neurotransmission and as an aid in smoking cessation is unknown. As such, there are a number of side effects that may present serious health risks to users, particularly those with an indication of schizophrenia, alcohol use, and seizures. Chantix, a nicotinic acetylcholine receptor partial agonist, is selective to the α4 nAchR subunit. Accordingly, there is a need in the art for compositions and methods for controlling nicotine addiction.

Nicotine, the main alkaloid found in tobacco, is the key addictive component that drives continued use through activation of neuronal nicotinic acetylcholine receptors (nAchR) (1-6). The molecular mechanism underlying nicotine addiction remains largely unclear; although nAchRs, N-methyl-D-aspartate (NMDA) glutamate receptor and cyclic AMP-response element binding protein (CREB) have all been shown to play important roles in the nicotine related reward pathway (2). In particular, the NMDA receptor has been demonstrated to play an important role in the development of sensitization for a variety of drugs including nicotine and alcohol. Therefore, there is a need to delineate the relationship between nAchR and NMDAR and provide agents and methods to modulate this relationship.

### SUMMARY OF THE INVENTION

The present invention provides compositions and methods for modulating nicotinic receptor function, N-methyl-D-aspartate (NMDA) receptor function, or both.

The present invention also provides compositions and methods for modulating the heterodimerization of α7 nicotinic acetylcholine receptors (nAChRs) and N-methyl-D-aspartate (NMDA) glutamate receptors. In particular, the compositions and methods prevent the heterodimerization of the nAchR and NMDAR.

According to an embodiment of the present invention there is provided a method for modulating nicotinic receptor function in a mammal in need of such treatment comprising administering an amount of an agent that disrupts, prevents, or inhibits the heterodimerization of the α7 neuronal nicotinic acetylcholine receptor and the N-methyl-D-asparate glutamate receptor.

In one embodiment, the agent is an antibody that binds to an amino acid sequence that is at least 80% identical to the IL2 of the α7 nAchR (SEQ ID NO: 1) or NMDAR[CT] (SEQ ID NO: 8).

In another embodiment, the amino acid sequence is identical to the sequence of the IL2 of the α7 nAchR (SEQ ID NO: 1) or NMDAR[CT] (SEQ ID NO: 8).

In a further embodiment, the antibody is fused to a protein transduction domain.

In a yet another embodiment, the agent is a nucleic acid encoding a polypeptide of between about 7 and about 150 amino acids comprising an amino acid sequence that is at least 80% identical to the sequence of α7 nAchR (SEQ ID NO: 1) or the sequence of NMDAR[CT] (SEQ ID NO: 8).

In yet a further embodiment, the nucleic acid encodes an amino acid sequence that is identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄ ; SEQ ID NO:8)).

In another embodiment, the nucleic acid is fused to a protein transduction domain.

In a further embodiment, the nucleic acid further encodes a protein transduction domain and the protein transduction domain is fused to the polypeptide.

In yet another embodiment, the agent is a polypeptide of between about 7 and about 150 amino acids comprising an amino acid sequence that is between about 80% and about 100% identical to sequence of α7 nAchR (SEQ ID NO: 1) or the sequence of NMDAR[CT] (SEQ ID NO: 8).

In yet a further embodiment, the polypeptide comprises an amino acid sequence that is identical to a sequence selected from the group consisting of α7_{IL2} of nAchR: R₃₁₆-R₄₆₉ (SEQ ID NO: 1), α7_{IL2} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄ ; SEQ ID NO:8)). The present invention also contemplates polypeptides and nucleic acids which encode polypeptides or fragments thereof that are 80% identical to 100% identical to NR1-1a_{CT}: E₈₃₄-S₉₃₈ (SEQ ID NO:7) or a fragment thereof.

In yet another embodiment the methods described hereinabove further comprise a protein transduction domain fused to the polypeptide.

In one particular embodiment of the invention, the method is for treating addiction or craving, for example, but not limited to, drug addiction or craving, nicotine addiction or craving, alcohol addition or craving or any combination thereof.

According to a further embodiment of the present invention there is provided a method for decreasing α7 nicotinic acetylcholine receptor (nAChRs) and N-methyl-D-aspartate (NMDA) glutamate receptor heterodimerization in a cell or tissue expressing α7 nAchR and NMDAR comprising administering an agent to the cell or tissue that inhibits the heterodimerization of α7 nAchR and NMDAR. The method may be an in-vitro method or an in-vivo method.

In one embodiment, the polypeptide comprises an amino acid sequence that is between about 80% and 100% identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-}(L₃₃₆-F₃₄₂; SEQ ID NO:5), or NMDAR[CT] (NR2A_{CT} (D₁₃₅₀-V₁₄₆₄ SEQ ID NO:8)).

In another embodiment, the polypeptide comprises an amino acid sequence that is identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄ SEQ ID NO:8)).

In yet another embodiment, the polypeptide further comprises a protein transduction domain or a carrier.

In yet further embodiment, the protein transduction domain is selected from the group consisting of TAT and SynB1/3Cit.

According to a further embodiment of the present invention there is a provided a nucleic acid encoding a polypeptide of between 7 and 150 amino acids comprising an amino acid sequence that is between about 80% identical and 100% identical to the sequence α7 nAchR (SEQ ID NO: 1) or NMDAR[CT] peptide (SEQ ID NO: 8).

In one embodiment, the polypeptide comprises an amino acid sequence that is between 80% and 100% identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄ ; SEQ ID NO:8)).

In another embodiment, the polypeptide comprises an amino acid sequence that is identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂, SEQ ID NO:5), or NMDAR[CT] (NR2A_{CT} (D₁₃₅₀-V₁₄₆₄; SEQ ID NO:8)).

In a further embodiment, the nucleic acid further encodes a protein transduction domain or carrier and the protein transduction domain or carrier is fused to the polypeptide.

In yet another embodiment, the protein transduction domain is selected from the group consisting of TAT, and SynB1/3Cit.

According to another embodiment of the present invention there is provided a kit that comprises: a) one or more proteins as described above or herein; b) one or more nucleic acids as described above or herein; c) one or more diluents, delivery vehicles, pharmaceutically acceptable excipients or a combination thereof; d) one or more devices for delivering polypeptides or nucleic acids to a solution, cell, cell culture, tissue, organ or subject; and e) instructions for using any component in the kit or practicing the method, or any combination thereof.

In one embodiment the kit comprises: a) a polypeptide comprising an amino acid sequence selected from the IL2 region of the α7 nAchR (SEQ ID NO:1) or fragment thereof; b) a polypeptide comprising an amino acid sequence selected from NMDAR[CT](SEQ ID NO:8) or a fragment thereof; c) a nucleic acid capable of expressing a polypeptide comprising the IL2 region of α7 nAchR amino acid sequence (SEQ ID NO: 1) or a fragment thereof; d) a nucleic acid capable of expressing a polypeptide comprising the NMDAR[CT] peptide (SEQ ID NO: 8) or a fragment thereof; e) one or more diluents, delivery vehicles, pharmaceutically acceptable excipients or a combination thereof; f) one or more devices for delivering polypeptides or nucleic acids to a solution, cell, cell culture, tissue, organ or subject; and g) instructions for using any component in the kit or practicing the method, or any combination thereof.

In a further non-limiting embodiment, there is provided a polypeptide or a nucleic acid encoding a polypeptide that is 80% to 100% identical to SEQ ID NO:7, or a fragment thereof.

The present invention also provides a method as described above, a polypeptide as described above, a nucleic acid as described above, a kit as described above, or any combination thereof wherein the polypeptide comprises the TAT amino acid sequence at the N-terminus of the polypeptide or peptide. Preferably, the TAT sequence is attached to the polypeptide or peptide via a peptide bond.

This summary of the invention does not necessarily describe all features of the invention.

### DETAILED DESCRIPTION OF THE DRAWINGS

These and other features of the invention will become more apparent from the following description in which reference is made to the appended drawings wherein:

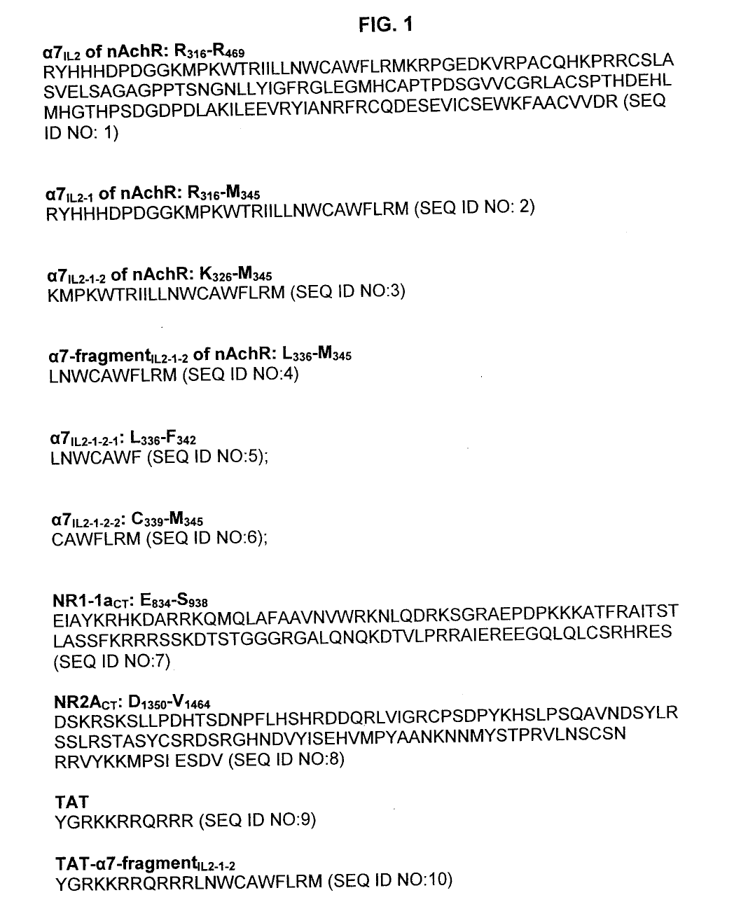

FIG. 1 shows the polypeptide constructs and amino acid sequences for α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), α7_{IL2-1-2-2}: C₃₃₉-M₃₄₅ (SEQ ID NO:6), NR1-1a_{CT}: E₈₃₄-S₉₃₈ (SEQ ID NO:7) and NMDAR[CT] (NR2A_{CT} (D₁₃₅₀-V₁₄₆₄ ; SEQ ID NO:8));

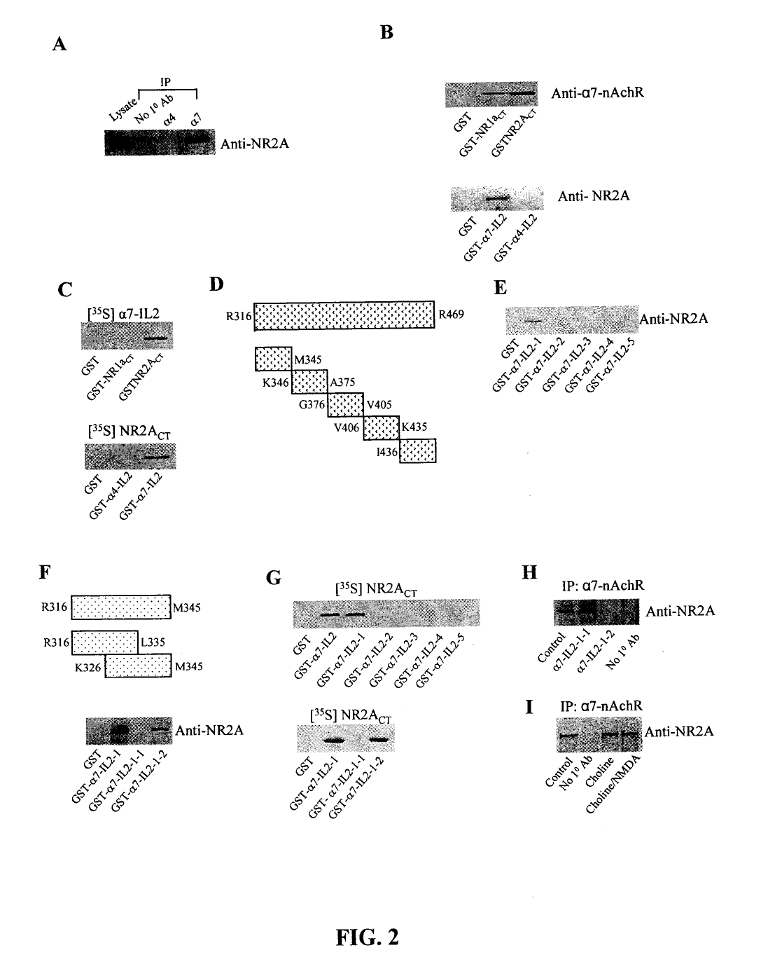

FIG. 2 shows the direct interaction between NMDARs and α7-nAchR. (A) Co-immunoprecipitation of NR2A subunit of NMDAR from solubilized rat hippocampal tissue with the α7-nAchR, but not α4-nAchR. (B) Western blots of hippocampal α7-nAchR (top), NR2A subunit of NMDAR (bottom) after affinity precipitation by GST-NR1a_{CT} (E₈₃₄-S₉₃₈), GST-NR2A_{CT} (D₁₃₅₀-V₁₄₆₄), and GST-α7_{IL2 (R}₃₁₆-R₄₆₉) respectively, but not GST-α4_{IL2}: V₃₃₂-K₅₉₅ or GST alone. (C) In vitro Binding Assay revealing direct binding of GST-NR2A_{CT} to [³⁵S]-α7_{IL2} (top), GST-α7_{IL2}, but not GST-α4_{IL2} to [³⁵S]-NR2A_{CT}. (D) Schematic representation of the generated α7_{IL2-1} to α7_{IL2-5} sub-fragments. (E) Western blot of hippocampal NR2A after affinity precipitation by GST-α7_{IL2-1} fragment, but not by other GST fused fragments or GST alone. (F) Schematic representation of the generated α7_{IL2-1-1} (R316-L335) and α7_{IL2-1-2} (K326-M345) sub-fragments (top). Western blot of hippocampal NR2A after affinity precipitation by GST-α7_{IL2-1-2}, but not by GST-α7_{IL2-1-1} or GST alone (bottom). (G) In vitro Binding Assay revealing direct binding of GST-α7_{IL2}, GST-α7_{IL2-1}, GST-α7_{IL2-1-2} to [³⁵S]-NR2A_{CT}. (H) Inhibition of the association between NMDAR and α7-nAchR upon the addition of α7_{IL2-1-2} peptide. (I) Pretreatment of hippocampal tissue with choline significantly increased the co-immunoprecipitation of NR2A with the α7-nAchR with no significant difference by NMDA/choline cotreatment;

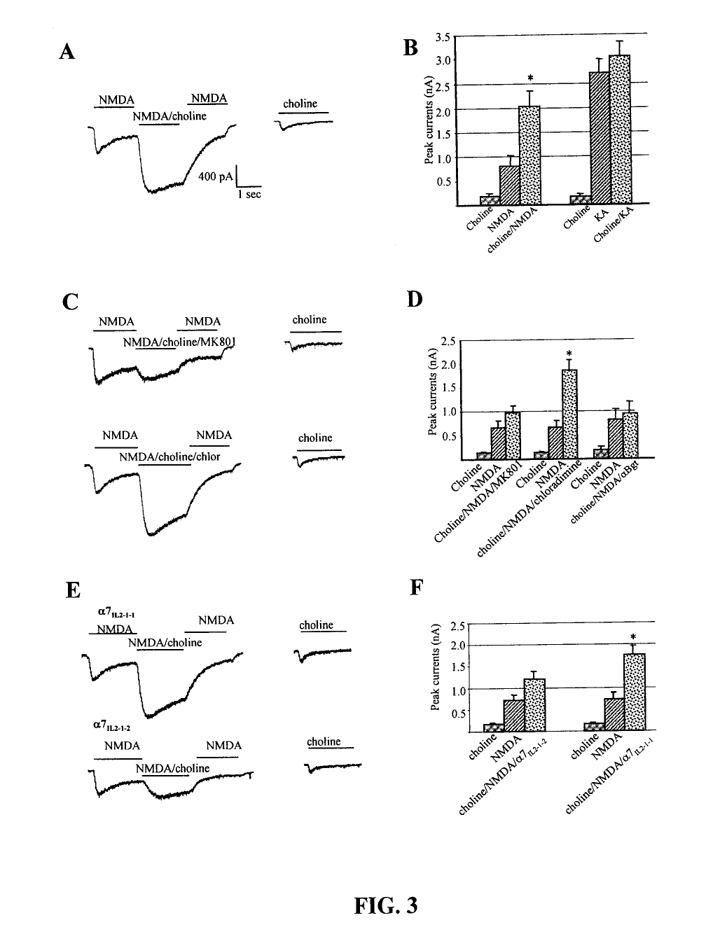

FIG. 3 shows the choline induced synergistic effect on NMDAR currents through the α7-nAchR/NMDAR direct protein-protein interaction. (A) Co-application of 1 mM choline with 50 μM NMDA/10 μM glycine produced a synergistic effect that display a significantly larger current compared to the current induced by NMDA/Glycine alone (n=43 of 47 cells, P<0.01). (B) The choline induced synergistic effect is specific to NMDAR-mediated currents since no such an effect was detected on currents induced by 100 μM kainic acid. (C, D) The choline-induced synergistic effect is significantly inhibited by simultaneous application NMDAR channel blocker MK-801 (10 μM) (n=8, p<0.05), but not the nAChR channel blocker chlorisondamine (20 μM). Furthermore, pretreatment of the neurons with the α7 nAChR specific antagonist α-Bungarotoxin for 40 minutes inhibited the choline-induced synergistic effect. (E, F) The choline-induced synergistic effect is significantly inhibited by intracellular application of α7_{IL2-1-2} interfering peptide, but not α7_{IL2-1-1} peptide (10 μM) (choline/NMDA: 1202.7±182.1 pA; NMDA: 910.5±130.8, n=6, p>0.05). Cells were held at −70 mV, 20 mM bicuculline, 1 mM strychnine, 0.5 TTX, 1 mM glycine were included in the extracellular solution;

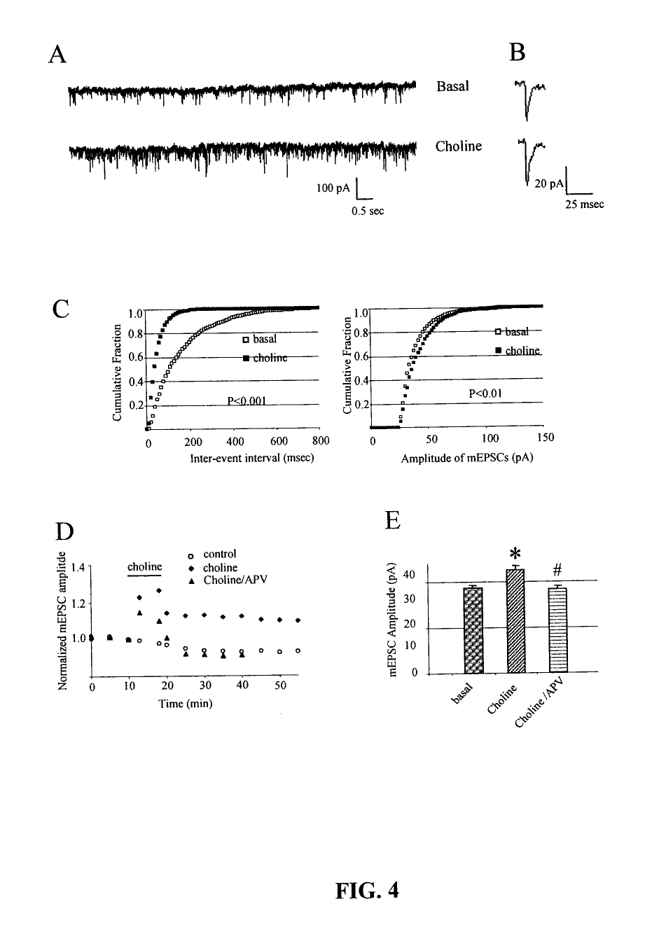

FIG. 4 shows the choline induced upregulation of NMDAR-dependent LTP of mEPSCs in cultured hippocampal neurons. (A) Examples of continuous recordings from individual neurons 5 minutes before (Basal) and 30 minutes after a 8-minute stimulation of neurons with 1 mM choline. (B) Single events taken from the basal and choline traces, respectively showing that the amplitude of mEPSCs was increased by choline application. (C) Cumulative fraction plots for mEPSCs inter-event intervals and amplitudes obtained 5 minutes before (Basal) and 30 minutes after choline (8 min, 1 mM). (D) mEPSC amplitudes are normalized to the values from the initial 10 min and plotted over time. Treatment of neurons with choline (8 min, 1 mM) significantly increased the amplitude of the mEPSCs over the time course of recordings; an effect can be abolished by NMDAR antagonist, AP5 (100 μM). (E) Amplitude histogram summarizes data from groups of individual neurons treated with glycine (200 μM; 3 min) in the absence or presence of choline (1 mM) or choline/AP5 (100 μM). Responses obtained 30 min after glycine treatment (26.5+/−2.3 pA), 30 min after choline treatment (31.4+/−2.7 pA, n=6, *p<0.01) and 30 minutes after coapplication of choline/APV (25.9+/−2.0 pA n=3, #p<0.05, paired t-test);

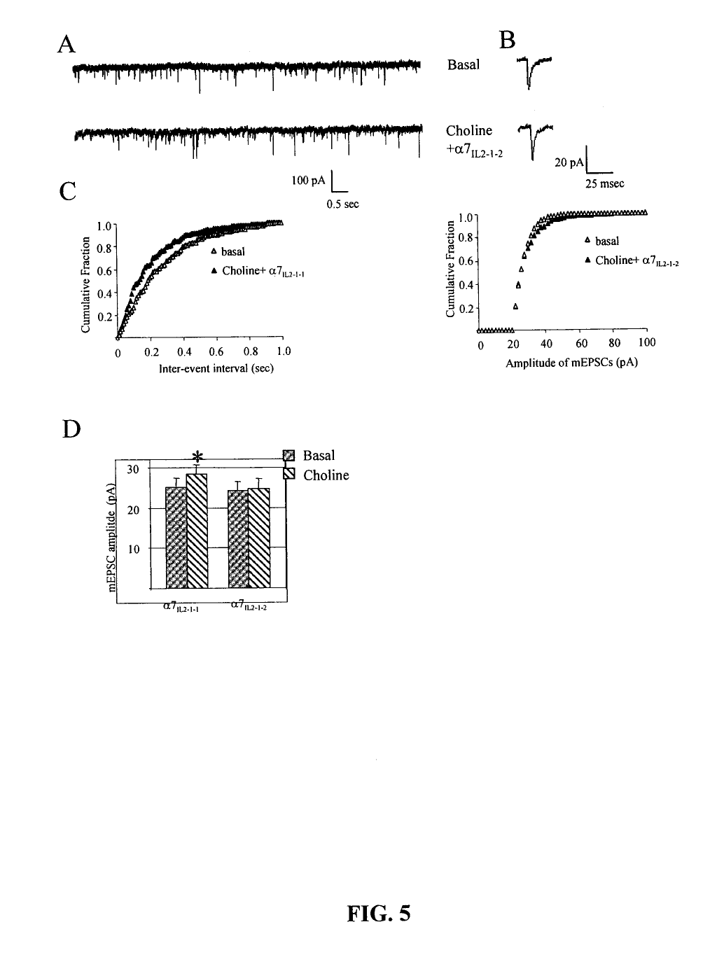

FIG. 5 shows that application of α7_{IL2-1-2} peptide blocked choline induced upregulation of mEPSC of LTP in hippocampal primary culture. (A) Examples of continuous recordings from individual neurons 40 minute after intracellular application of α7_{IL2-1-2} peptide (10 μM) (SEQ ID NO: 4) with/without the presence of choline (1 mM, 8 min). (B) Single events taken from the basal and choline traces after intracellular application of α7_{IL2-1-2} peptide (SEQ ID NO:4), showing that choline application failed to increase the amplitude of mEPSCs. (C) Cumulative fraction plots for mEPSCs inter-event intervals and amplitudes obtained 5 minutes before (Basal) and 30 minutes after choline (8 min, 1 mM) with the presence of α7_{IL2-1-2} peptide intracellularly. (D) Amplitude histogram summarizes data from groups of individual neurons treated with glycine (200 μM; 3 min) in the absence or presence of choline (1 mM) with the intracellular application of α7_{IL2-1-1} peptide and α7_{IL2-1-2} peptide, respectively. Responses obtained 30 min after glycine treatment (basal) and 30 min after choline treatment (choline). α7_{IL2-1-1} peptide did not block the enhancing effect of choline on the mEPSC amplitude (basal: 25.2+/−2.1 pA; choline: 28.4+/−2.4 pA, n=4, * p<0.01, paired t-test) while choline failed to upregulate mEPSC amplitude with the presence of α7_{IL2-1-2} peptide (basal: 24.2+/−2.0; choline: 25.1+/−2.3 pA, n=6, p>0.05, paired t-test);

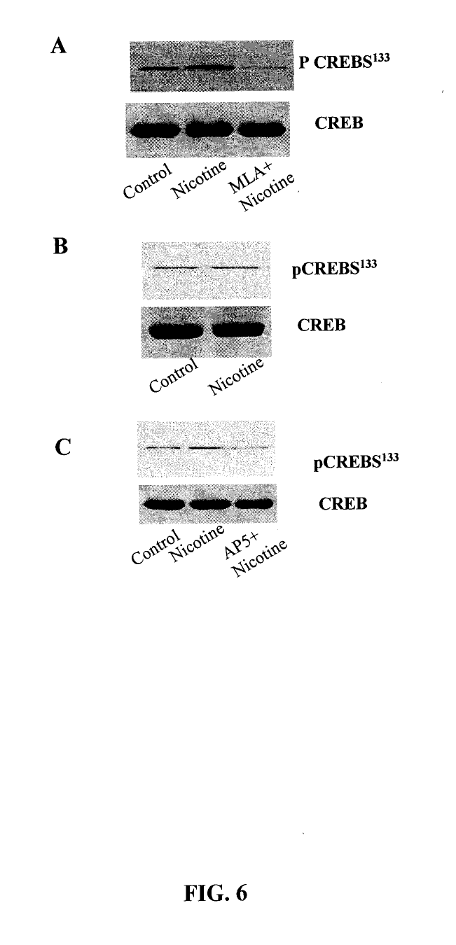

FIG. 6 shows that in cultured hippocampal neurons, (A) activation of nAChR with 1 mM nicotine (20 min) significantly enhances CREB activity; which can be blocked by MLA. (B) 1 mM nicotine failed to enhance CREB activity in HEK-293 cells expressing α7 nAChR only, suggesting that other proteins may be involved in the nicotine induced activation of CREB. (C) 1 mM nicotine significantly enhances CREB activity in HEK-293 cells co-expressing α7 nAChR and NMDAR NR1/NR2A subunits, suggesting an important role of NMDAR in this process;

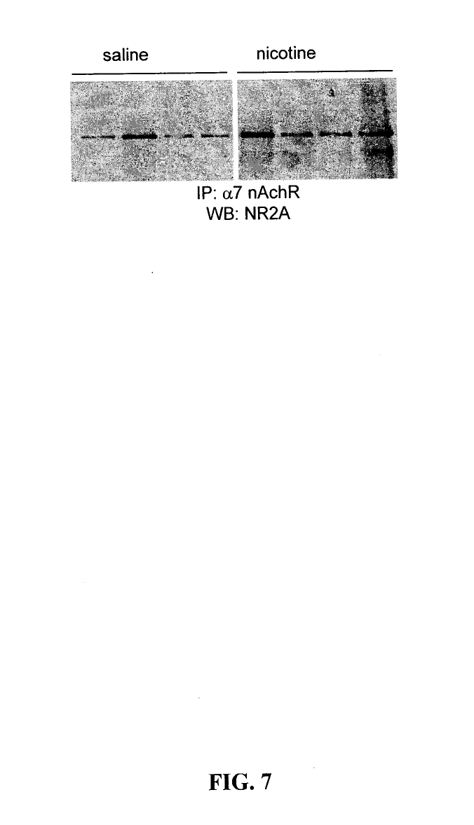

FIG. 7 shows coimmunoprecipitation of NR2A with α7-nAchR reveals an increase in the association of α7-NR2A in the hippocampus of rats chronically treated for 7 days with nicotine (6 mg/kg/day) compared to saline controls;

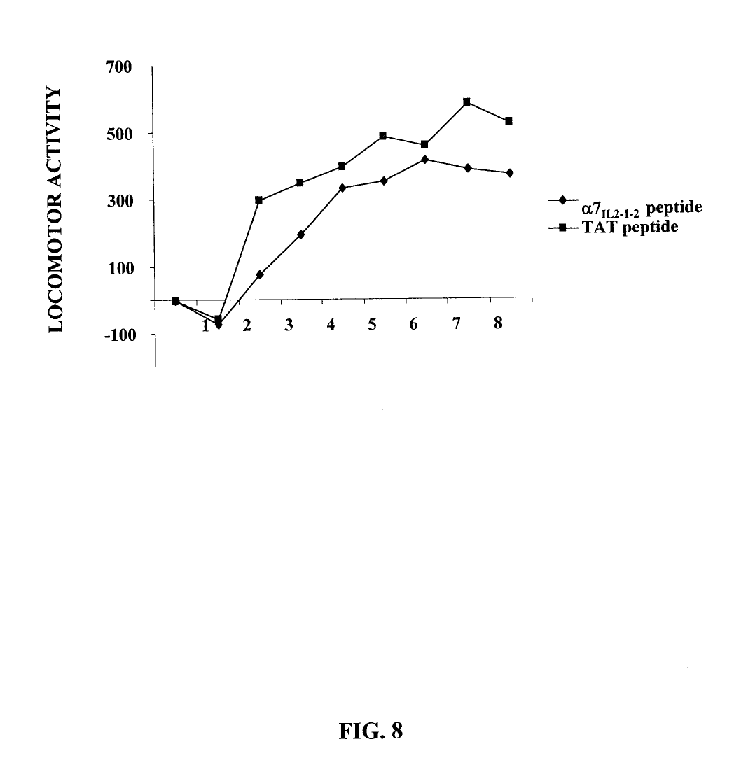

FIG. 8 shows the effect of the TAT-α7_{IL2-1-2} polypeptide on the development of sensitization of nicotine. Intracerebral ventricular injection (ICV) pretreatment with TAT-α7_{IL2-1-2} polypeptide (40 nmol) delays the development of sensitization to the motor activating effects of nicotine. TATα7_{IL2-1-2} peptide or vehicle control (TAT) was given 30 min before nicotine. Nicotine (0.35 mg/kg, sc) was administered every second day for a period of 6 days. Significant differences between TAT-α7_{IL2-1-2} peptide and control (p<0.05) were observed on day 2 and 3 of nicotine treatment. N=6 and 7 rats for control and TAT-α7_{IL2-1-2} peptide treatment group, respectively.

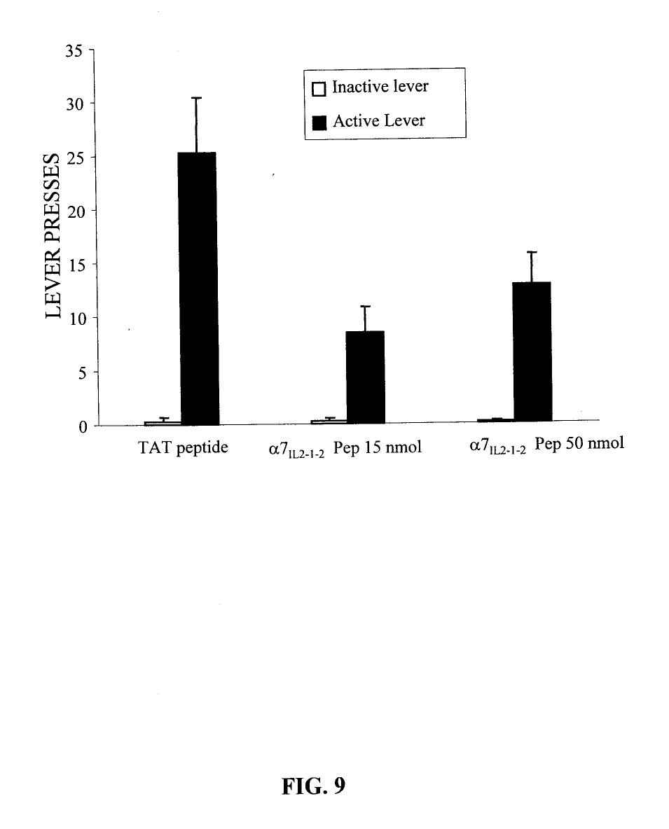

FIG. 9 shows the effect of interfering peptide TAT-α7_{IL2-1-2} on cue induced reinstatement of alcohol seeking intracerebral ventricular injection (ICV) of peptide significantly (P<0.005 for 50 nmol and p<0.02 for 15 nmol, n=11) blocks relapse to alcohol induced by re-exposure to cues previously associated with alcohol self-administration in an animal model. Peptide was delivered over a period of 1 minute, 60 minutes before the animals are tested for reinstatement.

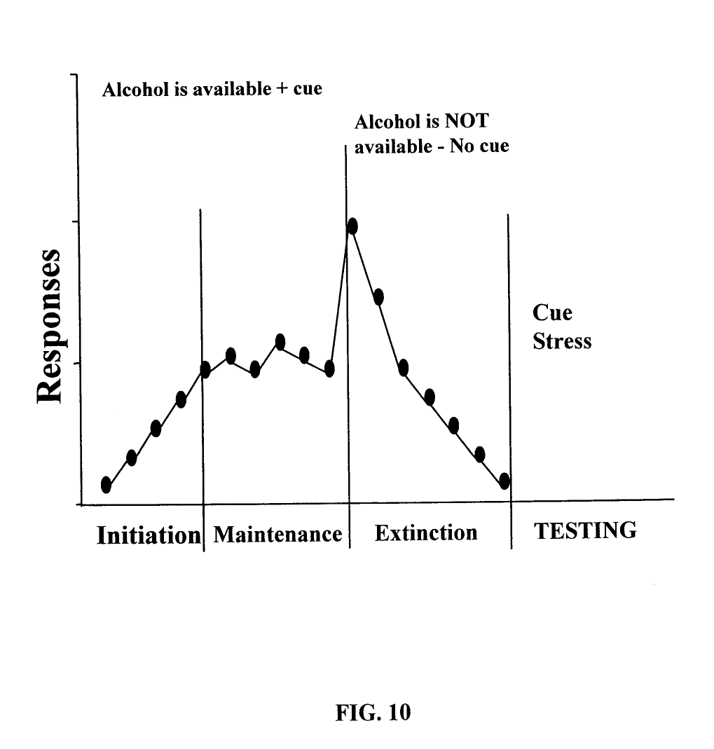

FIG. 10 shows typical results expected for an animal test model of relapse: Animals are first trained to self-administer drug (alcohol or nicotine) by pressing a lever, during this phase each alcohol (or nicotine) delivery is paired with light+tone cue; when alcohol (or nicotine) self-administration is stabilized, extinction is carried out (during extinction pressing on the lever has no consequence. No alcohol is administered). In time the animals stop pressing the lever as they do not get alcohol (or nicotine). Testing for relapse then takes place by reintroducing the cue previously paired with alcohol or nicotine delivery. Relapse is defined by an increase in response on the lever which is previously paired with alcohol delivery.

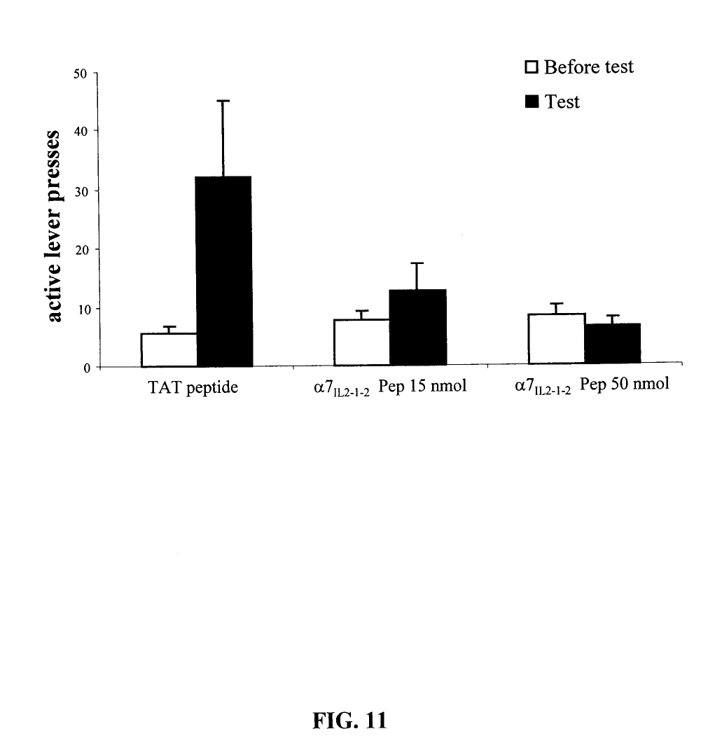

FIG. 11 shows the effect of interfering peptide TAT-α7_{IL2-1-2} on cue induced reinstatement of nicotine seeking. Relapse is a cardinal feature of drug dependence and exposure to cues previously associated with drug taking is a potent factor in promoting relapse to drug use. To determine whether TAT-α7_{IL2-1-2} interfering peptide can block relapse to nicotine, we examined its ability to block cue-induced reinstatement of nicotine seeking. As shown, re-exposure to cues previously associated with nicotine self-administration reinstated nicotine seeking as shown by an increase in responding on the active lever previously associated with nicotine delivery. Pretreatment with interfering peptide TAT-α7_{IL2-1-2} 15 nmol or 50 nmol administered by intraventricular route 1 hr prior to testing blocked reinstatement of nicotine seeking (relapse). (N=9 animals per dose). Similar blockade of reinstatement of alcohol seeking (relapse) was also observed following pretreatment with TAT-α7_{IL2-1-2} interfering peptide in a similar manner as that employed for nicotine study (N=11 per dose).

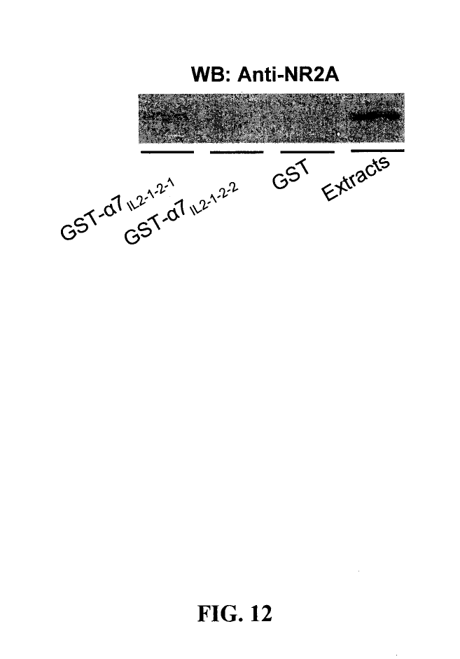

FIG. 12 shows the results of Western blot analysis in which NR2A receptor is immunoprecipitated by GST-α7_{IL2-1-2-1} (L336-F342; SEQ ID NO:5) in detergent extracts of rat hippocampus, but not GST-α7_{IL2-1-2-2} (C339-M345; SEQ ID NO:6) or GST alone.

### DETAILED DESCRIPTION OF THE INVENTION

The present invention relates to compositions and methods for modulating nicotinic receptor function. More specifically, the present invention relates to compositions and methods for modulating the heterodimerization of α7 nicotinic acetylcholine receptors (nAChRs) and N-methyl-D-aspartate (NMDA) glutamate receptors.

The following description is of a preferred embodiment.

Here we report that activation of α7 nAchR facilitated NMDAR-dependent long-term potentiation (LTP) and significantly enhanced CREB activation in cultured hippocampal neurons. α7 nAchR activation facilitates the formation of a protein complex with the NR2A subunit of the NMDAR. Disrupting the α7 nAchR-NR2A interaction diminishes the α7 nAchR induced up-regulation of NMDAR-dependent LTP and the activation of CREB in cultured hippocampal neurons. Thus, our results reveal a previously unappreciated cellular signaling pathway underlying activation of α7 nAchR and provide novel targets against which new therapeutics may be developed to combat diseases involving, for example, but not limited to α7 nAchR/NMDAR LTP and enhanced CREB activation. Since nicotinic receptors are believed to be involved in the pleasant effect of cigarettes described by smokers, and NMDA have long been shown to be involved in the pathophysiology of learning and addiction, disrupting the heterodimerization of α7 nAchR and NMDA, attempts to address both the desire and addiction aspects associated with smoking and other addictions.

According to the present invention, there is provided a method for decreasing nicotinic receptor function comprising inhibiting heterodimerization of the α7 nAchR and the NMDA glutamate receptor. In addition, according to the present invention there is provided a method for modulating nicotinic receptor function in a mammal in need of such treatment comprising administering an amount of an agent that disrupts, prevents, or inhibits the heterodimerization of the α7 nAchR and the NMDA glutamate receptor. For the purpose of the present application, the term “disrupt” includes disrupting an existing association, inhibiting or generally preventing the heterodimerization of the α7 nAchR and the NMDA glutamate receptor. Furthermore, there is provided a method for decreasing α7 nicotinic acetylcholine receptor (nAChRs) and N-methyl-D-aspartate (NMDA) glutamate receptor heterodimerization in a cell or tissue expressing α7 nAchR and NMDAR comprising administering an agent to the cell or tissue that inhibits the heterodimerization of α7 nAchR and NMDAR.

Our results show that activation of the α7 nAchR by an agonist causes heterodimerization of the α7 nAchR and the NMDA glutamate receptor. Heterodimerization of the two receptors by the binding of an agonist to the α7 nAchR results in an increase in the whole-cell current compared to activation of only the NMDA receptor alone. The protein complex formed by the heterodimerization of the α7 nAchR and NMDA receptor is mediated through the second intracellular loop (IL2) of the α7 nAchR and the carboxyl tail (CT) of the NR2A subunit of the NMDA receptor. Therefore, inhibiting the heterodimerization of α7 nAchR and the NMDA glutamate receptor may be accomplished by disrupting the binding of these two subunits. For example, various agents directed to these one or both of regions may used to disrupt the heterodimerization of the two receptors.

By “agent” it is meant any small molecule chemical compound, polypeptide, nucleic acid, or any combination thereof that can modulate nicotinic receptor mediated neurotransmission. By “modulate nicotinic receptor mediated neurotransmission” it is meant increasing nicotinic receptor mediated neurotransmission or decreasing nicotinic receptor mediated neurotransmission, for example, but not wishing to be limiting in any manner, by disrupting α7 nAchR/NMDA heterodimerization. A polypeptide may be of any length unless otherwise specified and includes, for example and without limitation, antibodies, enzymes, receptors, transporters, α7 nAchR receptor, NMDA, α7 nAchR fragment or derivative, or NMDA fragment or derivative. A fragment is any polypeptide or nucleic acid that is shorter than its corresponding naturally occurring polypeptide or nucleic acid, respectively. A derivative is any polypeptide or nucleic acid that is altered with respect to a reference polypeptide or nucleic acid, respectively, and includes, for example fragments or mutants. It is to be understood that the agent does not comprise a full length naturally occurring α7 nicotinic acetylcholine receptor or N-methyl-D-aspartate receptor, or a naturally occurring allelic variant thereof.

Accordingly, the present invention provides a polypeptide of less than 150 amino acids comprising an amino acid sequence that is at least 80% identical to the sequence of IL2 of α7 nAchR (SEQ ID NO: 1) or a fragment thereof, or the sequence of NMDA[CT] (SEQ ID NO:8) or a fragment thereof. In a preferred embodiment, the polypeptide is between about 7 and about 150 amino acids, for example, but not limited to 7, 8, 9, 10, 11, 12, 13, 14, 15, 17, 19, 20, 25, 30, 35, 40, 45, 50, 55, 60, 65, 70, 75, 80, 90, 100, 110, 120, 130 and 140 amino acids. In an alternate embodiment, the polypeptide is between about 15 and about 150 amino acids. However, it is to be understood that the size of the peptide may be defined by a range of any two of the values listed above. Also, in an alternate embodiment, which is not meant to be limiting in any manner, the present invention contemplates polypeptides as defined above which comprises more than 150 amino acids.

The present invention also provides a nucleic acid encoding polypeptides as defined above. For example, but not wishing to be limiting in any manner, the present invention contemplates a nucleic acid encoding a polypeptide of between about 7 and less than 150 amino acids, for example, but not limited to between 10 and 149 amino acids, between 10 and 140 amino acids, between 15 and 149 amino acids or between 15 and 140 amino acids and that encodes an amino acid sequence that is at least 80% identical to the sequence of IL2 of α7 nAchR (SEQ ID NO:1) or the sequence of NMDA (SEQ ID NO:8). In an alternate embodiment, the present invention contemplates nucleic acids or nucleotide sequences as described above but that encode more than 150 amino acids.

By “percent identical” or “percent identity”, it is meant one or more than one nucleic acid or amino acid sequence that is substantially identical to a coding sequence or amino acid sequence of peptides that can modulate nicotinic receptor mediated neurotransmission. By “substantially identical” is meant any nucleotide sequence with similarity to the genetic sequence of a nucleic acid of the invention, or a fragment or a derivative thereof. The term “substantially identical” can also be used to describe the similarity of polypeptide sequences. For example, nucleotide sequences or polypeptide sequences that are at least 80%, 85%, 90%, 92%, 95%, 96%, 97%, 98% or 99% identical to the α7 nAchR or NMDAR coding sequence, or the encoded polypeptide, respectively, or fragments or derivatives thereof, and still retain ability to affect α7 nAchR/NMDAR heterodimerization or modulate nicotinic receptor mediated neurotransmission are contemplated.

The present invention also contemplates an NMDA glutamate receptor binding polypeptide comprising an amino acid sequence selected from the IL2 region of the α7 nAchR (SEQ ID NO:1).

Fragments of the full-length IL2 region of the α7 nAchR are also capable of inhibiting the heterodimerization of α7 nAchR and the NMDA glutamate receptor. For example, a fragment based on the amino acid sequence from K₃₂₆-M₃₄₅ of α7 nAchR could be used to inhibit the heterodimerization of α7 nAchR and NMDA. Furthermore, a polypeptide based on L₃₃₆-M₃₄₅ of α7 nAchR could also be used to inhibit heterodimerization.

The present invention also contemplates polypeptides having an amino acid sequence that comprises between about 80% to 100% sequence identity, for example, but not limited to 80%, 81%, 82%, 83%, 84%, 85%, 86%, 87%, 88%, 89%, 90%, 91%, 92%, 93%, 94%, 95%, 96%, 97%, 98%, 99% or 100% identity to the amino acid sequences described above. Further, the polypeptides may be defined as comprising a range of sequence identities defined by any two of the values listed above.

It is also contemplated that the α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL-2-1-2-1} (L₃₃₆-F₃₄₂; SEQ ID NO:5), or NMDAR[CT] (NR2A_{CT} (D₁₃₅₀-V₁₄₆₄; SEQ ID NO:8)) or fragment thereof may comprise part of a fusion protein, for example, but not limited to a polypeptide that further comprises a heterologous polypeptide or protein, for example, a carrier protein, a protein transduction domain or the like. For example, but not wishing to be limiting in any manner, the polypeptide of the present invention may be fused to a protein transduction domain to facilitate transit across lipid bilayers or membranes, for example, but not limited to as described in U.S. Publication 2002/0142299, U.S. Pat. No. 5,804,604, U.S. Pat. No. 5,747,641, U.S. Pat. No. 5,674,980, U.S. Pat. No. 5,670,617, and U.S. Pat. No. 5,652,122; PCT publication W001/15511, US Publication 2004/0209797, PCT Publication W099/07728 ,US Publication 2003/0186890, all of which are herein incorporated by reference.

By protein transduction domain it is meant a sequence of nucleic acids that encode a polypeptide, or a sequence of amino acids comprising the polypeptide, wherein the polypeptide facilitates localization to a particular site, for example a cell or the like, or it may facilitate transport across a membrane or lipid bilayer. The polypeptides and nucleic acids of the present invention may be fused to a protein transduction domain to facilitate transit across lipid bilayers or membranes.

Many polypeptides and nucleic acids do not efficiently cross the lipid bilayer of the plasma membrane, and therefore enter into cells at a low rate. However, there are certain naturally occurring polypeptides that can transit across membranes independent of any specific transporter. Antennapedia (Drosophila), TAT (HIV) and VP22 (Herpes) are examples of such polypeptides. Fragments of these and other polypeptides have been shown to retain the capacity to transit across lipid membranes in a receptor-independent fashion. These fragments, termed protein transduction domains, are generally 10 to 27 amino acids in length, possess multiple positive charges, and in several cases have been predicted to be amphipathic. Polypeptides and nucleic acids that are normally inefficient or incapable of crossing a lipid bilayer, can be made to transit the bilayer by being fused to a protein transduction domain.

U.S. Publication 2002/0142299 (which is incorporated herein by reference) describes a fusion of TAT with human beta-glucuronidase. This fusion protein readily transits into various cell types both in vitro and in vivo. Furthermore, TAT fusion proteins have been observed to cross the blood-brain-barrier. Frankel et al. (U.S. Pat. No. 5,804,604, U.S. Pat. No. 5,747,641, U.S. Pat. No. 5,674,980, U.S. Pat. No. 5,670,617, and U.S. Pat. No. 5,652,122; which are incorporated herein by reference) have also demonstrated transport of a protein (beta-galactosidase or horseradish peroxidase) into a cell by fusing the protein with amino acids 49-57 of TAT.

PCT publication WO01/15511 (which is incorporated herein by reference) discloses a method for developing protein transduction domains using a phage display library. The method comprises incubating a target cell with a peptide display library and isolating internalized peptides from the cytoplasm and nuclei of the cells and identifying the peptides. The method further comprised linking the identified peptides to a protein and incubating the peptide-protein complex with a target cell to determine whether uptake is facilitated. Using this method a protein transduction domain for any cell or tissue type may be developed. US Publication 2004/0209797 (which is incorporated herein by reference) shows that reverse isomers of several of the peptides identified by the above can also function as protein transduction domains.

PCT Publication W099/07728 (which is incorporated herein by reference) describes linearization of protegrin and tachyplesin, naturally occurring as a hairpin type structure held by disulphide bridges. Irreversible reduction of disulphide bridges generated peptides that could readily transit cell membranes, alone or fused to other biological molecules. US Publication 2003/0186890 (which is incorporated herein by reference) describes derivatives of protegrin and tachyplesin that were termed SynB1, SynB2, SynB3, etc. These SynB peptides were further optimized for mean hydrophobicity per residue, helical hydrophobic moment (amphipathicity), or beta hydrophobic moment. Various optimized amphipathic SynB analog peptides were shown to facilitate transfer of doxorubicin across cell membranes. Further, doxorubicin linked to a SynB analog was observed to penetrate the blood-brain-barrier at 20 times the rate of doxorubicin alone.

The protein transduction domains described in the preceeding paragraphs are only a few examples of the protein transduction domains available for facilitating membrane transit of small molecules, polypeptides or nucleic acids. Other examples are transportan, W/R, AlkCWK18, DipaLytic, MGP, or RWR. Still many other examples will be recognized by persons skilled in the art

A protein transduction domain and an agent of the present invention may be placed together in sufficient proximity and maintained together for a sufficient time to allow the protein transduction domain to influence pharmaceutical product performance of the agent. Contemplated associations of protein transduction domain and agent include, for example and without limitation: non-covalent associations such as electrostatic interactions, hydrogen bonding, ionic bonds or complexes, Van der Weals bonds; covalent linkages such as conventional methods of cross-linking; linkages that are activated, in vitro and/or in vivo by electromagnetic radiation; any covalent bond such as a peptide bond; any biochemical interaction known to protein biochemists, such as biotin/streptavidin, nickel/Histidine, glutathione/glutathione-S-transferase, or antigen/antibody; physical associations within matrix structures or encapsulating systems; etc.

The present invention provides an agent that may be any small molecule chemical compound, polypeptide, nucleic acid, or any combination thereof that can inhibit or modulate α7nAchR heterodimerization with NMDAR by disrupting α7nAchR/NMDAR heterodimerization. Accordingly, the present invention provides a polypeptide of about 7 to less than about 150 amino acids, preferably 10 to 149 amino acids, more preferably 15 to 140 amino acids and comprising an amino acid sequence that is at least 80% identical, for example, but not limited to 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of IL2 of α7nAchR (SEQ ID NO:1) or the sequence of NMDAR[CT] (SEQ ID NO: 8). The present invention also provides a nucleic acid encoding a polypeptide of about 7 to less than about 150 amino acids, preferably about 10 to about 149 amino acids, more preferably about 15 to about 140 amino acids and comprising an amino acid sequence that is at least 80% identical, for example, but not limited to 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of IL2 of α7nAchR (SEQ ID NO:1) or the sequence of NMDAR[CT] (SEQ ID NO: 8). The polypeptide or nucleic acid may optionally be fused to a protein transduction domain, for example, but not limited to as described herein.

It is also contemplated that any one of the polypeptides of the present invention may be attached either covalently or non-covalently to a non-protein substrate or molecule, for example, but not limited to polyethylene glycol (PEG), dextran or polydextran bead or the like, a support such as, but not limited to a multi-well plate, coverslip, array, micro-chip or the like. It is also contemplated that the polypeptide, non-protein substrate, molecule or any combination thereof may be labeled, for example with a purification tag, a radioactive or fluorescent group, enzyme or the like.

The present invention also provides nucleic acids encoding the polypeptides as described above. In an embodiment of the present invention which is not meant to be limiting, there is provided a nucleic acid encoding a polypeptide comprising the IL2 of α7nAchR amino acid sequence (SEQ ID NO:1) or variations thereof. Preferably, but not wishing to be limiting in any manner, the present invention provides a nucleic acid encoding α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄; SEQ ID NO:8)) A nucleic acid encoding the TAT protein transduction domain attached to the polypeptide at the C-terminus, or the N-terminus, optionally including a spacer group of amino acids is also contemplated. Furthermore, a nucleic acid sequence encoding the polypeptide NMDAR[CT] (SEQ ID NO:8) is also contemplated.

The present invention also contemplates compositions comprising one or more of the polypeptides and/or nucleic acids of the present invention. The compositions may comprise one or more diluents, delivery vehicles, excipients, for example, but not limited to pharmaceutically acceptable excipients as would be known in the art, buffers, media, solvents, solutions, carriers or the like. Such components alone or in any combination may provide a dosage form for using or administering the polypeptides or nucleic acids of the present invention to a solution, cell, cell culture, tissue, organ or subject, for example, but not limited to a human subject.

To determine whether a nucleic acid exhibits identity with the sequences presented herein, oligonucleotide alignment algorithms may be used, for example, but not limited to a BLAST (GenBank URL: www.ncbi.nlm.nih.gov/cgi-bin/BLAST/, using default parameters: Program: blastn; Database: nr; Expect 10; filter: default; Alignment: pairwise; Query genetic Codes: Standard(1)), BLAST2 (EMBL URL: http://www.embl-heidelberg.de/Services/ index.html using default parameters: Matrix BLOSUM62; Filter: default, echofilter: on, Expect:10, cutoff: default; Strand: both; Descriptions: 50, Alignments: 50), or FASTA, search, using default parameters. Polypeptide alignment algorithms are also available, for example, without limitation, BLAST 2 Sequences (www.ncbi.nlm.nih.gov/blast/bl2seq/bl2.html, using default parameters Program: blastp; Matrix: BLOSUM62; Open gap (11) and extension gap (1) penalties; gap x_dropoff: 50; Expect 10; Word size: 3; filter: default).

An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. for at least 1 hour (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. for at least 1 hour (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, but not wishing to be limiting, stringent conditions are selected to be about 5° C. lower than the thermal melting point for the specific sequence at a defined ionic strength and pH.

A polypeptide of the invention can be synthesized in vitro or delivered to a cell in vivo by any conventional method. As a representative example of an in vitro method, the polypeptide may be chemically synthesized in vitro, or may be enzymatically synthesized in vitro in a suitable biological expression system. As a representative example of an in vivo method, a DNA, RNA, or DNA/RNA hybrid molecule comprising a nucleotide sequence encoding a polypeptide of the invention is introduced into an animal, and the nucleotide sequence is expressed within a cell of an animal.

The nucleotide sequence may be operably linked to regulatory elements in order to achieve preferential expression at desired times or in desired cell or tissue types. Furthermore, as will be known to one of skill in the art, other nucleotide sequences including, without limitation, 5′ untranslated region, 3′ untranslated regions, cap structure, poly A tail, translational initiators, sequences encoding signalling or targeting peptides, translational enhancers, transcriptional enhancers, translational terminators, transcriptional terminators, transcriptional promoters, may be operably linked with the nucleotide sequence encoding a polypeptide (see as a representative example “Genes VII”, Lewin, B. Oxford University Press (2000) or “Molecular Cloning: A Laboratory Manual”, Sambrook et al., Cold Spring Harbor Laboratory, 3rd edition (2001)). A nucleotide sequence encoding a polypeptide or a fusion polypeptide comprising the polypeptide may be incorporated into a suitable vector. Vectors may be commercially obtained from companies such as Stratagene or InVitrogen. Vectors can also be individually constructed or modified using standard molecular biology techniques, as outlined, for example, in Sambrook et al. (Cold Spring Harbor Laboratory, 3rd edition (2001)). A vector may contain any number of nucleotide sequences encoding desired elements that may be operably linked to a nucleotide sequence encoding a polypeptide or fusion polypeptide comprising a protein transduction domain. Such nucleotide sequences encoding desired elements, include, but are not limited to, transcriptional promoters, transcriptional enhancers, transcriptional terminators, translational initiators, translational terminators, ribosome binding sites, 5′ untranslated region, 3′ untranslated regions, cap structure, poly A tail, origin of replication, detectable markers, afffinity tags, signal or target peptide. Persons skilled in the art will recognize that the selection and/or construction of a suitable vector may depend upon several factors, including, without limitation, the size of the nucleic acid to be incorporated into the vector, the type of transcriptional and translational control elements desired, the level of expression desired, copy number desired, whether chromosomal integration is desired, the type of selection process that is desired, or the host cell or the host range that is intended to be transformed.

As described herein, and unless clearly indicated otherwise, the term “mini-gene” means the expression product of a nucleic acid or nucleotide sequence encoding and capable of expressing a polypeptide in a cell. For example, but not wishing to be considered limiting in any manner, a mini-gene includes a nucleic acid or nucleotide sequence encoding and capable of expressing the polypeptide comprising the IL2 of α7nAchR amino acid sequence (SEQ ID NO:1) or NMDAR[CT] (SEQ ID NO: 8) in a cell. In an alternate embodiment, the mini-gene comprises a nucleic acid or nucleotide sequence encoding and capable of expressing the polypeptide comprising the α7 of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M_{345;} SEQ ID NO:3), α7-fragment_{IL2-1-2} of nAchR (L₃₃₆-M₃₄₅; SEQ ID NO:4), α7_{IL2-1-2-1} , (L₃₃₆-F₃₄₂; SEQ ID NO:5), or NMDAR[CT] (NR2A_{CT} (D₁₃₅₀-V₁₄₆₄ SEQ ID NO:8)) in a cell. Each of these mini-genes could be attached to a sequence that expresses an adaptor protein that could assist in the stabilization, transfer and/or solubility of the desired protein.

The DNA, RNA, or DNA/RNA hybrid molecule may be introduced intracellularly, extracellularly into a cavity, interstitial space, into the circulation of an organism, orally, or by any other standard route of introduction for therapeutic molecules and/or pharmaceutical compositions. Standard physical methods of introducing nucleic acids include, but are not limited to, injection of a solution comprising RNA, DNA, or RNA/DNA hybrids, bombardment by particles covered by the nucleic acid, bathing a cell or organism in a solution of the nucleic acid, or electroporation of cell membranes in the presence of the nucleic acid.

A nucleic acid may be introduced into suitable eukaryotic cells ex vivo and the cells harbouring the nucleic acid can then be inserted into a desired location in an animal. A nucleic acid can also be used to transform prokaryotic cells, and the transformed prokaryotic cells can be introduced into an animal, for example, through an oral route. Those skilled in the art will recognize that a nucleic acid may be constructed in such a fashion that the transformed prokaryotic cells can express and secrete a polypeptide of the invention. Further, a nucleic acid may also be inserted into a viral vector and packaged into viral particles for efficient delivery and expression.

The polypeptides of the present invention or the nucleic acids encoding the polypeptides of the present invention may be formulated into any convenient dosage form as would be known in the art. The dosage form may comprise, but is not limited to an oral dosage form wherein the agent is dissolved, suspended or the like in a suitable excipient such as but not limited to water or saline. In addition, the agent may be formulated into a dosage form that could be applied topically or could be administered by inhaler, or by injection either subcutaneously, into organs, or into circulation. An injectable dosage form may include other carriers that may function to enhance the activity of the agent. Any suitable carrier known in the art may be used. Also, the agent may be formulated for use in the production of a medicament. Many methods for the productions of dosage forms, medicaments, or pharmaceutical compositions are well known in the art and can be readily applied to the present invention by persons skilled in the art.

The present invention also contemplates a method of modulating nicotinic receptor function comprising: administering a polypeptide comprising an amino acid sequence selected from the IL2 region of the α7 nAchR (SEQ ID NO:1) or a fragment thereof to a cell, tissue of subject in need thereof. The method may be practiced in vitro or in vivo. In an embodiment wherein the method is practiced in vivo, the method may be practiced in a human subject. The human subject may have a reliance, dependence or addiction to tobacco, thus the method could be used as a smoking cessation therapeutic or part of a smoking cessation program. Alternatively, the human subject may have a reliance, dependence or addiction to alcohol, thus the method could be used as an alcohol cessation therapeutic or part of an alcohol cessation program. In a further embodiment, the human subject may have a reliance, dependence or addiction to drugs, thus the method could be used as an drug addiction/cessation therapeutic or part of an drug addiction/cessation program.

Our results show that administration of α7_{IL2-1-2} peptide prior to nicotine exposure, delays the acquisition of nicotine sensitization but not the final level of sensitization attained. These results indicate that 1) the α7_{IL2-1-2} peptide is behaviorally active and 2) the effect is consistent with its action on NMDA receptors. The results provided herein also indicate that the polypeptides as described herein may be useful in treating and/or preventing alcohol addiction or relapse, or drug addiction and relapse.

The invention also provides a method of modulating nicotinic receptor function comprising: administering a nucleic capable of expressing a polypeptide comprising the IL2 region of α7 nAchR amino acid sequence (SEQ ID NO: 1) or NMDAR[CT] (SEQ ID NO:8) or a fragment thereof to a cell, cell culture, tissue or subject expressing α7 nAchR and NMDA glutamate receptor. The method may be practiced in vitro or in vivo. In an embodiment wherein the method is practiced in vivo, the method may be practiced in a human subject. The human subject may have a reliance, dependence or addiction to tobacco, alcohol or drugs, thus the method could be used a smoking cessation therapeutic or part of a smoking cessation program, an alcohol cessation therapeutic or part of an alcohol cessation program, a drug cessation therapeutic or drug cessation program, or a combination thereof.

Also provided by the present invention is a method of modulating nicotinic receptor function comprising: administering a polypeptide comprising an amino acid sequence selected from the IL2 region of the α7 nAchR (SEQ ID NO:1) or NMDAR[CT] (SEQ ID NO:8) or fragment thereof; or a nucleic capable of expressing a polypeptide comprising the IL2 region of α7 nAchR amino acid sequence (SEQ ID NO: 1) or a fragment thereof; to a cell or tissue or subject in need thereof. Accordingly, the method may be practiced in vitro or in vivo. In an embodiment wherein the method is practiced in vivo, the method may be practiced in a human subject. The human subject may have a reliance, dependence or addiction to tobacco, alcohol or drugs, thus the method could be used a smoking cessation therapeutic or part of a smoking cessation program, an alcohol cessation therapeutic or part of an alcohol cessation program, a drug cessation therapeutic or drug cessation program, or a combination thereof.

Also provided by the present invention is a kit that comprises: a) a polypeptide comprising an amino acid sequence selected from the IL2 region of the α7 nAchR (SEQ ID NO:1) or fragment thereof; b) a polypeptide comprising an amino acid sequence selected from NMDAR[CT](SEQ ID NO:8) or a fragment thereof; c) a nucleic acid capable of expressing a polypeptide comprising the IL2 region of α7 nAchR amino acid sequence (SEQ ID NO: 1) or a fragment thereof; d) a nucleic acid capable of expressing a polypeptide comprising the NMDAR[CT] peptide (SEQ ID NO: 8) or a fragment thereof; e) one or more diluents, delivery vehicles, pharmaceutically acceptable excipients or a combination thereof; f) one or more devices for delivering polypeptides or nucleic acids to a solution, cell, cell culture, tissue, organ or subject; and g) instructions for using any component in the kit or practicing any method as described herein, or any combination thereof. Further the kit may comprise any number of components that would be known to those skilled in the art.

The present invention will be further illustrated in the following examples:

### EXAMPLES

### Experimental Procedures

### Primary Hippocampal Neuron Culture

Primary cultures from hippocampus were prepared from fetal Wistar rats (embryonic day 17-19) on Cell+ (Sarstedt) culture dishes as previously described in (10). The cultures were used for experiments on 12-15 d after plating.

### Electrophysiology

Miniature excitatory postsynaptic currents (mEPSCs) were recorded from cultured hippocampal neurons 2 to 4 weeks days after plating under whole-cell patch clamp configuration (11). Electrodes (3-5 MΩ) were pulled from high lead pipettes (Corning 8161, Warner Instruments). Cells were voltage clamped at −70 mV. Access resistance is below 10 MΩ, recordings with access resistance varying more than 10% were rejected from analysis.

### Example 1

α7 nAchR Co-Immunoprecipitates with NMDA Glutamate Receptors.

To determine the existence of α7: NMDA receptor complexes, we examined if α7-nAchR can co-immunoprecipitate with NMDA receptors in rat hippocampal tissue. As depicted in FIG. 2A, immunoprecipitation of α7-nAchR resulted in the co-precipitation of the NMDA receptor NR2A subunit suggesting a physical association between α7-nAchR and NMDA receptors. Both the carboxyl tail (CT) of the NR1/NR2A subunits and the second intracellular loop (IL2) of α7-nAchR contain putative consensus sequences for receptor phosphorylation and potential binding sites for various proteins important for signalling [e.g. PSD-95, calmodulin] (24-25). To determine if the CT regions of NMDA receptors and the IL2 region of α7-nAchR are involved in the formation of α7-nAch: NMDA receptor complex, various glutathione-S-transferase (GST) fusion proteins, encoding the CT of the NR1a (GST-NR1-1a_{CT}: E₈₃₄-S₉₃₈), NR2A (GST-NR2A_{CT}: D₁₃₅₀-V₁₄₆₄ subunits or the IL2 of the α7 (GST-α7_{IL2}: R₃₁₆-R₄₆₉), α4 subunit of nAchR (GST-α4_{IL2}: V₃₃₂-K₅₉₅), were prepared and utilized in affinity purification assays. As shown in FIG. 2B (top panel), GST-NR1a_{CT} and GST-NR2A_{CT}, but not GST alone, could precipitate solubilized hippocampal α7-nAchR except for GST-NR1a_{CT} precipitated α7-nAchR with less efficiency compared to GST-NR2A_{CT}. Similarly, GST-α7_{IL2}, but not GST-α4_{IL2} or GST alone, precipitated solubilized hippocampal NR2A subunits indicating that the α7-nAch receptor can interact with NMDA receptors through its second intracellular loop, as illustrated in FIG. 2B (bottom panel).

### Example 2

IL2 Region of α7 nAchR Directly Binds with the NR2A Subunit of NMDAR

In vitro binding assay provided evidence that α7-nAchR and NR2A subunit can directly interact with each other. As shown in FIG. 2C (top panel), in vitro translated [³⁵S]-α7-IL2 probe hybridized with GST-NR2A_{CT} but not GST-NR1a_{CT} or GST alone, indicating a direct protein-protein interaction between the α7 subunit of nAchR and the NR2A subunit and an indirect interaction between α7 and NR1-1a subunit, which may be attributed to the interaction between NR1-1a with the NR2A subunit. Similarly, [³⁵S]-NR2A probe hybridized with GST-α7_{IL2} but not GST-α4_{IL2} or GST alone (FIG. 2C, bottom panel), confirming the specificity of the direct protein-protein interaction between α7-nAch and NR2A subunit of NMDA receptors.

### Example 3

Identification of Interaction Sites of the IL2 Region of α7 nAchR and the NR2A Subunit Complex

In order to delineate the region of the α7_{IL2} involved in the interaction with NR2A, five α7_{IL2} GST-fusion proteins (α7_{IL2-1}; R₃₁₆-M₃₄₅, α7_{IL2-2}: K₃₄₆-A₃₇₅, α7_{IL2-3}: G₃₇₆-V₄₀₅, α7_{IL2-4}: V₄₀₆-K₄₃₅, α7_{IL2-5}: I₄₃₆-R₄₆₉) were constructed (FIG. 2D) and utilized in affinity purification assays. As shown in FIG. 2E, only GST-α7_{IL2-1} was able to precipitate NR2A, confirming that the α7 subunit can interact with NR2A through its IL2 region R₃₁₆-M₃₄₅. Using a similar approach, α7_{IL2-1} was dissected into two smaller fragments α7_{IL2-1-1}: R₃₁₆-L₃₃₅ and α7_{IL2-1-2}: K₃₂₆-M₃₄₅ (FIG. 2F, top). Affinity purification assays identified amino acids K₃₂₆-M₃₄₅ as the specific region of α7 that forms protein complex with NR2A, as shown in FIG. 2F (bottom) where GST-α7_{IL2-1-2} was able to precipitate NR2A while GST-α7_{IL2-1-1} and GST alone failed to precipitate NR2A from solubilized rat hippocampal tissue. Consistent with the affinity purification experiments, in vitro translated [³⁵S]-NR2A CT probe hybridized only with GST-α7_{IL2-1} (FIG. 2G, top panel) and α7_{IL2-1-2} (FIG. 2G, bottom panel). Both GST-α7_{IL2-1-1} and GST-α7_{IL2-1-2} were designed with 10 amino-acid (K₃₂₆-L₃₃₅) overlapping region to avoid the possible disruption of the binding motif.

However, only GST-α7_{IL2-1-2} interacts with the NR2A CT, which implicates the L₃₃₆-M₃₄₅ region of IL2 of α7-nAchR as being important in the direct protein-protein interaction between α7-nAchR and the NR2A subunit of NMDA receptors. Furthermore, coimmunoprecipitation studies exhibited that preincubation with synthetic peptides containing sequences of α7_{IL2-1-2} decreased the α7-nAch/NR2A interaction compared to control peptide α7_{IL2-1-1} (FIG. 2H).

In order to better understand if a small polypeptide fragment of the L₃₃₆-M₃₄₅ amino acid sequence was important for the direct protein-protein interaction between α7-nAchR and the NR2A subunit of NMDA receptors, the GST-α7_{IL2-1-2-1} (L₃₃₆-F₃₄₂) and GST-α7_{IL2-1-2-2} (C₃₃₉-M₃₄₅) polypeptide fragments were prepared and tested (see FIG. 12). The L₃₃₆-F₃₄₂ polypeptide but not the C₃₃₉-M₃₄₅ polypeptide or GST alone resulted in immunoprecipitation of NR2A suggesting L₃₃₆-F₃₄₂ as being important in the direct protein-protein interaction between α7-nAchR and the NR2A subunit of NMDA receptors.

### Example 4

Agonist Binding to α7 nAchR Results in Heterodimerization with NMDAR

To examine the effect of α7-nAchR activation on the interaction between α7-nAchR and NMDA receptors, we examined the ability of antibody against NR2A subunit to co-precipitate with the α7-nAchR in hippocampal primary culture treated with 1 mM choline or choline/NMDA (50 μM). As shown in FIG. 21, activation of α7-nAchR by choline led to significant increase in α7-nAchR-NR2A interaction, with no significant effect by NMDA cotreatment, suggesting that the interaction is upregulated upon α7-nAchR activation.

### Example 5

α7 nAchR Activation Enables a Functional Modulation of NMDA Receptor Function

To assess whether the increase in the α7-nAchR/NMDA receptor physical interaction upon α7-nAchR activation enables a functional modulation of NMDA receptor function, we examined the effects of α7-nAchR activation on NMDA-receptor-mediated whole-cell currents in rat hippocampal primary cultures. As shown in FIG. 3A, co-application of 1 mM choline with 50 μM NMDA/10 μM glycine produced a significantly larger current compared to the current induced by NMDA/Glycine alone (choline/NMDA/glycine: 2036.3±317.2 pA; NMDA/glycine: 812.9±215.5 pA, n=43, p<0.05). The choline induced enhancement of current is specific to NMDA receptor since co-application of choline with 100 μM KA did not enhance whole-cell currents compared to KA treatment alone (FIG. 3B). It is difficult to differentiate whether the observed enhancement of whole current induced by co-application of choline with NMDA is mediated by nicotinic receptors or NMDA receptors since both receptors are cation ion channel that are permeable to calcium and sodium. However, the fact that the observed enhancement of whole cell current induced by co-application of choline with NMDA can be blocked by simultaneous application of the NMDA receptor channel blocker MK-801 (10 μM), but not with the nicotinic receptor open channel blocker chlorisondamine (20 μM) (FIG. 3C, D), suggesting the observed enhancement of whole cell currents may reflect the ion influx through NMDA receptor, but not nicotinic receptors. Furthermore, α7-nAChR specific antagonists α-bungarotoxin can abolish the choline induced upregulation of NMDA-mediated current (FIG. 3D), indicating the activation of α7-nAChR is required in this process.

### Example 6

Polypeptide Disruption of the Heterodimerization of α7 nAchR and NMDAR

We then investigated whether the α7-nAchR/NMDA direct coupling plays a role in the functional interaction between α7-nAchR and NMDA receptor. As shown in FIG. 3E, F, intracellular application of the α7-nAchR/NMDA coupling interfering peptide (α7_{IL2-1-2}, 10 μM), which has been shown to be able to disrupt the α7-nAchR/NMDA coupling (FIG. 3H), significantly blocked the choline-induced enhancement of NMDA-mediated whole cell current, while the control peptide, α7_{IL2-1-1} has no such effect suggesting that the direct binding of α7-nAchR to NMDA receptor NR2A subunit is obligatory for the functional modulation of NMDA receptors by the activation of α7-nAchR.

### Example 7

α7 nAchR Modulates Miniature Excitatory Postsynaptic Currents During LTP in Primary Cultures of Hippocampal Neurons

To determine whether activation of α7-nAchR is physiologically relevant in regulating synaptic strength, we examined the ability of α7-nAchR activation to modulate miniature excitatory postsynaptic currents (mEPSCs) during LTP in primary cultures of hippocampal neurons. mEPSCs in hippocampal neurons were recorded under whole-cell configuration. Similar to the electrically evoked EPSCs in CA1 neurons in hippocampal slices, mEPSCs can exhibit either LTP or LTD, depending on the way NMDAR is pharmacologically activated (11). In order to test if enhancement of NMDA receptor function could result in long-term potentiation in glutamatergic synaptic transmission, we delivered 1 mM choline focally to the soma and proximal dendritic areas up to 8 minutes. The cells were held at −70 mV to avoid potential interference by voltage-gated Ca⁺⁺ channels (12,13). As shown in FIG. 4A, C, choline application significantly enhanced the frequency of mEPSC of LTP, which might due to an enhanced transmitter release (14-19), or conversion of silent synapses to functional ones (20-21). While there is a more robust increase in current frequency, there is only a small and not significant increase in current amplitude (FIG. 4B, D and E), which may reflect the nature of LTP in primary culture and the recording paradigm (11,22,23).

### Example 8

Polypeptide Disruption of α7 nAchR Induction of LTP

To confirm that α7-nAchR-mediated induction of LTP is dependent on the interaction between α7-nAchR/NMDAR, we examined the ability of choline to induce LTP in the presence of the interfering peptide α7_{IL2-1-2}. As shown in FIG. 5A-G, intracellular application of α7_{IL2-1-2} peptide significantly attenuated choline-induced upregulation of mEPSCs of LTP with respect to current frequency, indicating a critical role for the α7-nAchR/NMDAR coupling in this process.

### Example 9

Activation of α7 nAChR Increases the Activity of CREB in a NMDAR Dependent Manner.

Given that CREB has been shown to play a critical role in learning/memory and drug addiction, we then tested whether activation of nAChR changes the activity of CREB. As shown in FIG. 6A, in cultured hippocampal neurons, activation of nAChR with 1 μM nicotine (20 min) significantly enhances CREB activity; which can be blocked by MLA, the α7 nAchR antagonist. To confirm that α7 nAChR is responsible for the observed activation of CREB, we measured CREB activity in HEK-293 cells expressing α7 nAChR. Nicotine failed to enhance CREB activity, suggesting that other proteins may be involved in the nicotine induced activation of CREB (FIG. 6B). Previous studies have suggested that NMDAR may be involved in the nicotine induced activation of CREB; therefore, we measured CREB activity in HEK-293 cells co-expressing α7 nAChR and NR1/NR2A subunits. As shown in FIG. 6C, nicotine significantly enhances CREB activity; suggesting the critical role of NMDAR in this process.

### Example 10

Chronic Nicotine Exposure Facilitates the α7 nAChR: NR2A Complex Formation.

Since α7 nAChR-NR2A coupling forms the molecular basis of nicotine addiction, chronic nicotine exposure should promote the α7 nAChR: NR2A complex formation. Thus, we pretreated rats with either nicotine or saline for 7 days subdermally (osmotic mini-pump, 6 mg/kg/day), and the expression of NR2A and α7 nAChR was examined in Western blot analysis. While there is no significant difference in the expression of NR2A or α7 nAChR in rat hippocampus between the saline and nicotine treatment (data not shown), the co-immunoprecipitation results indicated the α7 nAChR: NR2A complex formation is significantly enhanced in chronic nicotine exposured rat hippocampus compare to the control subjects (FIG. 7).

### Example 11

### Effects of Interfering Peptide (α7_{IL2-1-2}) on the Development of Sensitization/Tolerance to Nicotine and Alcohol.

Behavioral sensitization is the phenomenon that occurs when repeated administration of a drug results in an enhancement of a behavioural response to the drug over time. Tolerance refers to the attenuation of a particular drug response with repeated administration of the drug. These are two common features associated with repeated exposure to psychostimulants, nicotine and alcohol. These phenomena (sensitization/tolerance) have been regarded as learning processes and contribute to the development of drug dependence.

Since α7_{IL2-1-2} peptide interferes with the interaction between the α7 nicotinic receptor subunit and the NMDA receptor, we have conducted a study to evaluate its effect on the development of sensitization to the locomotor activating effects of nicotine. Administration of the peptide prior to nicotine, delays the acquisition of nicotine sensitization (FIG. 8). These results indicate that 1) α7_{IL2-1-2} peptide is behaviorally active and 2) the effect is consistent with its action on NMDA receptors.

### Example 12

### Methods for Testing Alcohol or Nictine Relapse

Rats were trained to self-administer alcohol or nicotine in operant chambers for about 6 (or 3 for nicotine) weeks. The operant chamber was equipped with 2 levers: an active and inactive lever. Pressing on the active lever resulted in a delivery of alcohol (or nicotine) whereas pressing on the inactive lever has no programmable consequences. The requirement for alcohol (or nicotine) delivery was increased from FR-1 (one press on the active lever leads to one delivery dose of alcohol) to FR-3 (3 presses are required for each delivery dose) for the last 2 weeks. During one our session, rats would obtain about 15 delivery doses of alcohol. This is equivalent to about 1 g/kg per hour or about 4-5 bottles of beer for an average person in one hour.

Extinction of drug self-administration was then carried out for 8-15 sessions (one session per day). During these sessions, pressing on the active lever did not lead to drug delivery, and the cue light and tone signaling drug delivery were disconnected. After 14-15 extinction sessions the number of lever presses on the active lever is reduced to less than 10-1 presses per hour comparing to about 70 presses when alcohol or nicotine was available.

Re-introducing the light and tone that are previously associated with alcohol or nicotine delivery leads to a significant increase in the number of lever presses on the active lever which indicates drug seeking or drug relapse. This is compatible to exposure to environmental cues that are/were associated with drug consumption (bar, smell of beer etc). The suppression of responding on the active lever by interfering peptide treatment α7_{IL2-1-2} following re-exposure to cues previously associated with alcohol or nicotine self-administration indicates that peptide is effective in blocking relapse to alcohol or nicotine in this model. It was concluded that intracerebral ventricular injection (ICV) of peptide significantly (P<0.005 for 10 and p<0.02 for 3 mM dose) blocks relapse to alcohol or nicotine induced by re-exposure to cues previously associated with alcohol or nicotine self-administration in an animal model.

The present invention has been described with regard to one or more embodiments. However, it will be apparent to persons skilled in the art that a number of variations and modifications can be made without departing from the scope of the invention as defined by the claims.

### REFERENCES

The references listed below as well as all references cited in the specification are incorporated herein by reference to the extent that they supplement, explain, provide a background for, or teach methodology, techniques and/or compositions employed herein.

1. Balfour, D J, Wright, A E, Benwell, M E and Birrell, C E, (2000). Behav Brain Res 113, pp. 73-83.

2. Dani, J A and Heinemann, S. (1996) Neuron 16, pp. 905-908.

3. Dani, J A, Ji, D and Zhou, F-M, (2001). Neuron 31, pp. 349-352.

4. Di Chiara, G, (2000). Eur J Pharmacol 393, pp. 295-314.

5. Schelling, T C, (1992). Science 255, pp. 430-433.

6. Stolerman, I P and Shoaib, M, (1991). Trends Pharmacol Sci 12, pp. 467-473.

7. Ausubel, et al. (eds), 1989, Current Protocols in Molecular Biology, Vol. 1, Green Publishing Associates, Inc., and John Wiley & Sons, Inc., New York, at p. 2.10.3.

8. Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.

9. “Genes VII”, Lewin, B. Oxford University Press (2000) or “Molecular Cloning: A Laboratory Manual”, Sambrook et al., Cold Spring Harbor Laboratory, 3rd edition (2001).

10. Lee et al, 2002a “Dual regulation of NMDA receptor functions by direct protein-protein interactions with the dopamine D1 receptor”. Cell 111, 219-230.

11. Lu et al., (2001), Neuron 29, pp. 243-254.

12. Fisher and Dani, (2000), Neuropharmacology 39, pp. 2756-2769.

13. Chang and Berg, (2001), Neuron 32, pp. 855-865.

14. McGehee et al., (1995), Science 269, pp. 1692-1696.

15. Gray et al., (1996), Nature 383, pp. 713-716.

16. Role and Berg, (1996), Neuron 16, pp. 1077-1085.

17. Albuquerque et al., (1997), J Pharmacol Exp Ther 280, pp. 1117-1136.

18. Wonnacott, (1997), Trends Neurosci 20, pp. 92-98.

19. Radicliffe and Dani, (1998), J Neurosci 18, pp. 7075-7083.

20. Isaac et al., (1995), Neuron 15, pp. 427-434.

21. Liao et al., (1995), Nature 375, pp. 400-444.

22. Fitzjohn et al., (2001), Neuropharmacology 41, pp. 693-699.

23. Sokolova et al., (2006), J Neurophysiol 95, pp. 2570-2590.

24. Lezcano et al., (2000) Science. Mar 3;287(5458):1660-4.

25. Lamey et al., (2001) J Biol Chem. Mar 15;277(11):9415-21.

1. A method for modulating nicotinic/NMDA receptor function in a mammal in need of such treatment comprising administering an amount of an agent that disrupts heterodimerization of α7 neuronal nicotinic acetylcholine receptors and N-methyl-D-asparate glutamate receptors.

2. The method of claim 1, wherein the agent is an antibody that binds to an amino acid sequence that is at least 80% identical to the IL2 of the α7 nAchR (SEQ ID NO: 1) and NMDAR[CT] (SEQ ID NO: 8).

3. The method of claim 2, wherein the amino acid sequence is identical to the sequence of the IL2 of the α7 nAchR (SEQ ID NO: 1) or NMDAR[CT] (SEQ ID NO: 8).

4. The method of claim 3, wherein the antibody is fused to a protein transduction domain.

5. The method of claim 1 , wherein the agent is a nucleic acid encoding a polypeptide of between about 7 and about 150 amino acids comprising an amino acid sequence that is at least 80% identical to the IL2 sequence of α7 nAchR (SEQ ID NO: 1) or the sequence of NMDAR[CT] (SEQ ID NO: 8).

6. The method of claim 5, wherein the polypeptide comprises an amino acid sequence that is identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL-2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄ ; SEQ ID NO:8)).

7. The method of claim 5, wherein the nucleic acid is fused to a protein transduction domain.

8. The method of claim 5, wherein the nucleic acid further encodes a protein transduction domain and the protein transduction domain is fused to the polypeptide.

9. The method of claim 1, wherein the agent is a polypeptide of between about 7 and about 150 amino acids comprising an amino acid sequence that is between about 80% and about 100% identical to the sequence of α7 nAchR (SEQ ID NO:1) or the sequence of NMDAR[CT] (SEQ ID NO: 8).

10. The method of claim 9, wherein the polypeptide comprises an amino acid sequence that is identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α_{IL2-1-2} of nAchR (K_{326-M}₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄; SEQ ID NO:8)).

11. The method of claim 9, further comprising a protein transduction domain fused to the polypeptide.

12. The method of claim 1, wherein the method is for treating addiction or craving.

13. A polypeptide of between about 7 and about 150 amino acids comprising an amino acid sequence that is between about 80% and 100% identical to the sequence of α7 nAchR (SEQ ID NO: 1) or NMDAR[CT] peptide (SEQ ID NO: 8).

14. The polypeptide of claim 13, comprising an amino acid sequence that is between about 80% and 100% identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α 7_{IL2-1-2-1} (L₃₃₆-F₃₄₂; SEQ ID NO:5), or NMDAR[CT] (NR2A_{CT} (D₁₃₅₀-V₁₄₆₄ ; SEQ ID NO:8)).

15. The polypeptide of claim 13, comprising an amino acid sequence that is identical to a sequence selected from the group consisting of α7_{IL2} of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), or NMDAR[CT] (NR2A_{CT} (D₁₃₅₀-V₁₄₆₄ ; SEQ ID NO:8)).

16. The polypeptide of claim 13, further comprising a protein transduction domain.

17. The polypeptide of claim 16, wherein the protein transduction domain is selected from the group consisting of TAT and SynB1/3Cit.

18. A nucleic acid encoding a polypeptide of between 7 and 150 amino acids comprising an amino acid sequence that is between about 80% identical and 100% identical to the sequence α7 nAchR (SEQ ID NO: 1) or NMDAR[CT] peptide (SEQ ID NO: 8).

19. The nucleic acid of claim 18, wherein the polypeptide comprises an amino acid sequence that is between 80% and 100% identical to a sequence selected from the group consisting of α7_{IL2} of nAchR: R₃₁₆-R₄₆₉ (SEQ ID NO: 1), α7_{IL2-1} of nAchR (R₃I₆-M₃₄S″, SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), and NMDAR[CT] (NR2A_{CT} (D₁₃₅₀-V₁₄₆₄ ; SEQ ID NO:8)).

20. The nucleic acid of claim 19, wherein the polypeptide comprises an amino acid sequence that is identical to a sequence selected from the group consisting of α7_{IL2}of nAchR: R₃₁₆-R₄₆₉ (SEQ ID NO: 1), α7_{IL2-1} of nAchR (R₃₁₆-M₃₄₅; SEQ ID NO: 2), α7_{IL2-1-2} of nAchR (K₃₂₆-M₃₄₅; SEQ ID NO:3), α7-fragment_{IL2-1-2} of nAchR (L₃₃₆-M₃₄₅; SEQ ID NO:4), α7_{IL2-1-2-1} (L₃₃₆-F₃₄₂; SEQ ID NO:5), and NMDAR[CT] (NR2A_{CT} (D₁₃₅₀-V₁₄₆₄; SEQ ID NO:8)).

21. The nucleic acid of claim 18, wherein the nucleic acid further encodes a protein transduction domain and the protein transduction domain is fused to the polypeptide.

22. The nucleic acid of claim 21, wherein the protein transduction domain is selected from the group consisting of TAT, and SynB1/3Cit.

23. A method for decreasing α7 nicotinic acetylcholine receptor (nAChRs) and N-methyl-D-aspartate (NMDA) glutamate receptor heterodimerization in a cell or tissue expressing α7 nAchR and NMDAR comprising administering an agent to the cell or tissue that inhibits the heterodimerization of α7 nAchR and NMDAR.

24. A kit that comprises:
a) a polypeptide comprising an amino acid sequence selected from the IL2 region of the α7 nAchR (SEQ ID NO:1) or fragment thereof;
b) a polypeptide comprising an amino acid sequence selected from NMDAR[CT](SEQ ID NO:8) or a fragment thereof;
c) a nucleic acid capable of expressing a polypeptide comprising the IL2 region of α7 nAchR amino acid sequence (SEQ ID NO: 1) or a fragment thereof;
d) a nucleic acid capable of expressing a polypeptide comprising the NMDAR[CT] peptide (SEQ ID NO: 8) or a fragment thereof;
e) one or more diluents, delivery vehicles, pharmaceutically acceptable excipients or a combination thereof;
f) one or more devices for delivering polypeptides or nucleic acids to a solution, cell, cell culture, tissue, organ or subject; and
g) instructions for using any component in the kit or practicing any method as described herein.

25. A polypeptide or a nucleic acid encoding a polypeptide that is 80% to 100% identical to SEQ ID NO:7, or a fragment thereof.

26. The method of claim 1, wherein the polypeptide comprises TAT (YGRKKRRQRRR; SEQ ID NO:9) at the N-terminus of the polypeptide linked via a peptide bond.
